(19) United States
(12) Patent Application Publication
(10) Pub. No.: US 2014/0286979 A1
(43) Pub. Date: Sep. 25, 2014

(54) CANINE INFLUENZA RECOMBINANT VIRUS, PREPARATION METHOD THEREFOR AND APPLICATION THEREOF

(71) Applicants: Zejun Li, Shanghai (CN); Qiaoyang Teng, Shanghai (CN)

(72) Inventors: Zejun Li, Shanghai (CN); Qiaoyang Teng, Shanghai (CN)

(73) Assignee: SHANGHAI VETERINARY RESEARCH INSTITUTE, CAAS, Shanghai (CN)

(21) Appl. No.: 14/119,366

(22) PCT Filed: Apr. 26, 2012
(86) PCT No.: PCT/CN2012/074722

§ 371 Date: Mar. 31, 2014

(30) Foreign Application Priority Data
May 26, 2011 (CN) 201110139528.3

(51) Int. Cl. C12N 7/00; G01N 33/569

(52) U.S. Cl. CPC C12N 7/00; G01N 33/56983
USPC 424/186.1; 435/235.1; 435/5

(57) ABSTRACT

A canine influenza recombinant virus includes HA and NA genes of ZJCIV canine influenza virus as well as six internal genes PA, PB1, PB2, M, NP and NS of a PR8 virus. The nucleotide sequence of the HA gene is selected from the group consisting of: (1) a nucleotide sequence encoding an amino acid sequence of SEQ ID NO.1; (2) a nucleotide sequence encoding an amino acid sequence which has at least 98% sequence identity to the amino acid sequence of SEQ ID NO.1. The nucleotide sequence of the NA gene is selected from the group consisting of: (1) a nucleotide sequence encoding an amino acid sequence of SEQ ID NO.2; (2) a nucleotide sequence encoding an amino acid sequence which has at least 98% sequence identity to the amino acid sequence of SEQ ID NO.2.

### FIELD OF THE INVENTION

The present invention relates to the technical field of bioengineering, in particular to a canine influenza recombinant virus and a preparation method therefore and an application thereof.

### BACKGROUND OF THE INVENTION

Influenza A virus is a major infectious disease that poses a threat to human health. Influenza viruses possess strict host specificity, so even spreading of the same virus on different hosts is still restricted by the host factor. Back in 2004, it was first reported in the United States that a major canine influenza outbreak was caused by an H3N8 subtype canine influenza virus, and it is found through sequence analysis that this subtype canine influenza virus derives from evolution of an equine influenza virus. Subsequently, equine influenza in Australia was also followed by a canine influenza outbreak. A canine influenza outbreak caused by an H3N2 subtype canine influenza virus began in Korea in 2008, and it is found through sequence analysis that this H3N2 subtype canine influenza virus is an avian-origin virus, which is different from the equine-origin canine influenza viruses in the United States and Europe.

Several H3N2 subtype canine influenza viruses were in vivo isolated from virus-infected canines in South China from 2006 to 2007, and it is found through sequence analysis that these viruses are highly homologous with the isolated virus in Korea. Surveys on pet dogs' serums in South China found that 6.7% of these dog serums are positive for influenza. In 2010, a canine influenza virus was also isolated from canines in East China by our lab and named as A/canine/Zhejiang/01/2010 (H3N2 subtype, ZJCIV for short). It is found through complete genome sequence analysis for the ZJCIV virus that this virus is highly homologous with the canine influenza viruses from South China and the H3N2 canine influenza virus from Korea. According to animal infection experiments, the ZJCIV virus is infectious for canines, and could cause a canine disease that is manifested as loss of appetite, hyperpyrexia, cough, nasal drainage of purulent secretions and other symptoms, and pulmonary congestion, pulmonary hemorrhage and inflammatory exudate-filled pulmonary alveoli are found after dissection.

A canine influenza virus inactivated vaccine was successfully developed by Intervet Company in June 2009 and has been commercially available in the United States, however, there is a difference in antigenicity between the pandemic canine influenza virus in China and the pandemic canine influenza virus in the United States, these two viruses originate from different branches of an H3 influenza virus. Thus, development of vaccines against H3N2 subtype canine influenza virus pandemic strains is of important and practical significance to prevention and control for canine influenza.

### SUMMARY OF THE INVENTION

The technical problem to be solved by the present invention is to provide a canine influenza recombinant virus. The canine influenza recombinant virus comprises: HA and NA genes of ZJCIV canine influenza virus as well as six internal genes of a PR8 virus, and the recombinant virus can be prepared into vaccines that are effectively against H3N2 subtype canine influenza virus.

In addition, a preparation method for the canine influenza recombinant virus and an application of the canine influenza recombinant virus also need to be provided.

To solve the technical problems above, the present invention is conducted as follows:

In one aspect, the present invention provides a canine influenza recombinant virus, which comprises HA and NA genes of ZJCIV canine influenza virus as well as six internal genes PA, PB1, PB2, M, NP and NS of a PR8 virus.

The nucleotide sequence of the HA gene of the canine influenza virus is selected from the group consisting of:

(1) a nucleotide sequence encoding an amino acid sequence of SEQ ID NO.1;

(2) a nucleotide sequence encoding an amino acid sequence which has at least 98% sequence identity to the amino acid sequence of SEQ ID NO.1;

the nucleotide sequence of the NA gene of the canine influenza virus is selected from the group consisting of:

(1) a nucleotide sequence encoding an amino acid sequence of SEQ ID NO.2;

(2) a nucleotide sequence encoding an amino acid sequence which has at least 98% sequence identity to the amino acid sequence of SEQ ID NO.2.

Preferably, the HA gene of the canine influenza virus has a nucleotide sequence of SEQ ID NO.3, or the HA gene of the canine influenza virus has a sequence having at least 98% sequence identity to the nucleotide sequence of SEQ ID NO.3.

Preferably, the NA gene of the canine influenza virus has a nucleotide sequence of SEQ ID NO.4, or the NA gene of the canine influenza virus has a sequence having at least 98% sequence identity to the nucleotide sequence of SEQ ID NO.4.

In the present invention, the amino acid sequence having more than 98% of homology with the amino acid sequence of SEQ ID NO.1 comprises an amino acid sequences having the hemagglutinin (HA) activity of the ZJCIV canine influenza virus, derived from deletion, addition, insertion or substitution of one or more amino acids in the amino acid sequence of SEQ ID NO.1.

In the present invention, the amino acid sequence having more than 98% of homology with the amino acid sequence of SEQ ID NO.2 comprises an amino acid sequences having the neuraminidase (NA) activity of the ZJCIV canine influenza virus, derived from deletion, addition, insertion or substitution of one or more amino acids in the amino acid sequence of SEQ ID NO.2.

HA and NA are two important surface antigens of an influenza virus, and antigenic variation of this influenza virus refers mainly to variation of HA and NA, in particular, faster variation occurs in HA. Therefore, the HA gene of the ZJCIV canine influenza virus in the canine influenza recombinant virus of the present invention is a nucleotide sequence encoding the amino acid sequence of SEQ ID NO.1, or a nucleotide sequence encoding an amino acid sequence having more than 98% of homology with the amino acid sequence of SEQ ID NO.1; the NA gene of the ZJCIV canine influenza virus in the canine influenza recombinant virus of the present invention is a nucleotide sequence encoding the amino acid sequence of SEQ ID NO.2, or a nucleotide sequence encoding an amino acid sequence having more than 98% of homology with the amino acid sequence of SEQ ID NO.2.

In another aspect, the present invention further provides a preparation method for the canine influenza recombinant virus, the method comprising:

constructing recombinant plasmids comprising the HA and NA genes of the ZJCIV canine influenza virus respectively;

transfecting the recombinant plasmids of the HA and NA genes, and six plasmids comprising the internal genes PA, PB1, PB2, M, NP and NS of the PR8 virus respectively, to a 293T cell, and culturing the transfected cell;

inoculating the cultured cell supernatant to a chicken embryo, culturing the chicken embryo in an incubator for a proper time period to obtain chicken embryo allantoic fluid, detecting the hemagglutination condition of the allantoic fluid, and at the presence of hemagglutinin activity, determining the absence of unexpected variations by sequencing to obtain the canine influenza recombinant virus.

Preferably, the cultured cell supernatant is inoculated to a 9-day to 11-day chicken embryo, and the chicken embryo is cultured in a 37° C. incubator for 48-72 hours to obtain chicken embryo allantoic fluid.

PBD vectors are used as empty vectors in the recombinant plasmids.

Still in another aspect, the present invention further provides an application of the canine influenza recombinant virus for preventing or treating canine influenza.

Still in another aspect, the present invention further provides an influenza vaccine comprising the canine influenza recombinant virus.

Preferably, the influenza vaccine further comprises adjuvant.

Still in another aspect, the present invention further provides a method of preventing or treating canine influenza , the method comprising administering the influenza vaccine to a subject.

The canine influenza recombinant virus in the present invention can generate very high virus titer and hemagglutination titer on both a chicken embryo and an MDCK cell, and can be used as a good seed virus for developing canine influenza vaccines.

### BRIEF DESCRIPTION OF THE DRAWINGS

The present invention will be further described below in details with reference to the accompanying drawings and the embodiments.

FIG. 1 is an RT-PCR electrophoretogram of the HA and NA of the ZJCIV canine influenza virus in the embodiment 1 of the present invention;

FIG. 2 is a graph illustrating hemagglutination titers at different time after inoculation of the rescued recombinant virus and the ZJCIV to a chicken embryo in the embodiment 3 of the present invention;

FIG. 3 is a graph illustrating growth curve comparison of the rescued recombinant virus and the ZJCIV on the chicken embryo in the embodiment 3 of the present invention;

FIG. 4 is a graph illustrating hemagglutination titers at different time after inoculation of the rescued recombinant virus and the ZJCIV to an MDCK cell in the embodiment 3 of the present invention;

FIG. 5 is a graph illustrating growth curve comparison of the rescued recombinant virus and the ZJCIV on the MDCK cell in the embodiment 3 of the present invention.

### DETAILED DESCRIPTION OF THE INVENTION

In the embodiments described hereinafter, experimental methods in which specific conditions are unspecified are typically carried out under general conditions, e.g. the method described in Short Protocols in Human Genetics (Edited by Ausubel F. M., Kingston R. E., Seidman J. G. et al., Translated by MA, Xuejun, SHU, Yuelong. Beijing: Science Press, 2004).

One of the critical prerequisites for developing canine influenza virus vaccines is good seed virus, and the A/canine/Zhejiang/01/2010 virus (H3N2 subtype, ZJCIV for short) that is previously isolated in the lab has extremely low hemagglutination titer after amplification no matter on chicken embryos or on cells. Thus in the present invention, the primary antigen proteins, i.e. HA and NA genes, of the ZJCIV canine influenza virus, and the other six internal genes of the PR8 virus are recombined together, so as to rescue, by means of the reverse genetic system of the influenza virus, a canine influenza recombinant virus that can generate very high virus titer and hemagglutination titer on both a chicken embryo and a cell, and this recombinant virus can be used as a good seed virus for developing canine influenza vaccines.

### EXAMPLE 1

### Construction and Identification of the Recombinant Plasmids

b 1. PCR Amplification

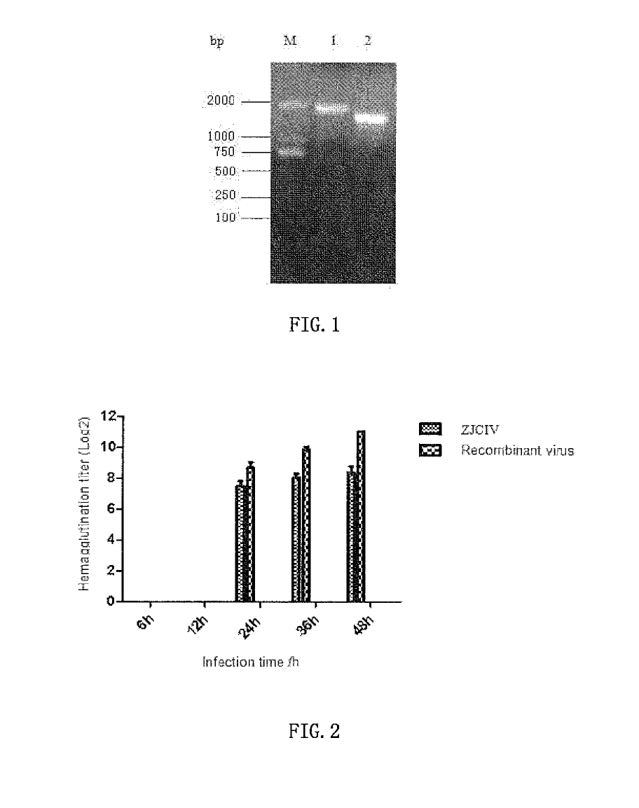

The total RNA of the canine influenza virus ZJCIV is extracted using Trizol (Invitrogen). With a 12-bp primer 5′-AGCAAAAGCAGG-3′ serving as the specific primer, first-strand cDNA is synthesized using Reverse Transcription System Kit (TakaRa) according to its instruction. The HA and NA of the fragment ZJCIV are respectively amplified by taking the resultant first-strand cDNA as the template and taking sapI-HA-up, sapI-HA-down and sapI-NA-up, sapI-NA-down as upstream and downstream primers (containing BspQl enzyme digestion sites, as shown in Table 1). The PCR amplification procedure is as follows: pre-degenerate at 94° C. for 5 minutes, enter the following cycle: degenerate at 94° C. for 45 seconds, anneal at 53° C. for 45 seconds and extend at 72° C. for 1 minutes and 45 seconds, complete 30 cycles, and finally, extend at 72° C. for 10 minutes. Meanwhile, a negative control with no template is established. At the end of reaction, PCR products undergo electrophoresis on 1.0% agarose gel. The results are shown in FIG. 1: two PCR strips appear, i.e. HA having a size of about 1700 bp and NA having a size of about 1400 bp, and destination fragments are consistent in size. In FIG. 1, M: DNA molecular weight marker; 1: ZJCIV HA PCR product; 2: ZJCIV NA PCR product.

TABLE 1
Universal Primers of the HA and NA Genes of the Influenza A Virus

| Primer Name | Primer Sequence |
|---|---|
| sapI-HA-up | CACACAgctcttctattAGCAAAAGCAGGGG (SEQ ID NO. 5) |
| sapI-HA-down | CACACAgctcttcggccAGTAGAAACAAGGGTGTTTT (SEQ ID NO. 6) |
| sapI-NA-up | CACACAgctcttctattAGCAAAAGCAGGAGT (SEQ ID NO. 7) |
| sapI-NA-down | CACACAgctcttcggccAGTAGAAACAAGGAGTTTTTT (SEQ ID NO. 8) |

2. Gel Cutting Recovery of the PCR Products

At the end of electrophoresis, the agarose gel of the destination DNA fragment is cut off from gel under ultraviolet light, and DNA is recovered by a DNA rapid recovery kit. The specific method is as follows: cut off the destination-DNA-containing agarose gel under a ultraviolet lamp, absorb all the liquid on the surface of the gel by tissue, cut the gel into pieces, put the gel pieces in a sterile 1.5 ml EP tube, add Buffer DE-A (liquid gel) the volume of which is 3 times as much as that of the gel (100 mg=100 ul), mix uniformly and then heat at 75° C., mix intermittently (for 2-3 minutes) until the blocky gel is completely molten (about 6-8 minutes); add Buffer DE-B (binding buffer) the volume of which is a half of that of the Buffer DE-A, and mix uniformly. Add isopropanol the volume of which is equal to that of the gel when the recovered DNA fragment is less than 400 bp. Transfer the mixed solution into a DNA preparation tube, centrifuge for 1 minute at a rate of 12000×g, and pour the waste solution in the collection tube. Place the preparation tube back into the collection tube, add 500 ul Buffer W1 (cleaning solution), centrifuge for 30 seconds at a rate of 12000×g, and pour the waste solution in the collection tube. Place the preparation tube back into the collection tube, add 700 ul Buffer W2 (desalting solution), centrifuge for 1 minute at a rate of 12000 ×g, pour the waste solution in the collection tube, and wash the preparation tube in the same way once again. Place the preparation tube back into the collection tube, and centrifuge in vacuum for 1 minute at a rate of 12000×g. Finally, place the preparation tube in the clean 1.5 ml EP tube, add 30 ul of de-ionized water to the center of a preparation membrane, stand for 1 minute under room temperature, centrifuge for 1 minute at a rate of 12000×g to elute DNA, and preserve the product at minus 20° C. for future use.

3. Enzyme Digestion, Ligation and Transformation

According to the instruction, the purified PCR products and the PBD vectors (preserved in the lab) are incubated at 50° C. for 1 hour under the action of BspQl restriction endonuclease (NEB). The enzyme-digested products of the destination fragments and the PBD plasmids are recovered using a gel extraction kit, 1 ul of T4 ligase buffer solution and 1 ul of T4 ligase (TakaRa) are added, the reaction system for ligation is 10 ul, and uniform mixing is carried out. Ligation is carried out overnight at 16° C. The ligation product is transformed to a competent cell JM109 (prepared in the lab), and the competent cell is then coated on an Amp-containing LB solid culture medium in a clean bench under aseptic conditions, followed by culture for 8-20 hours at 37° C.

4. Identification of the Recombinant Plasmids

A single colony on the LB solid culture medium is picked out and then placed in the test tube having Amp-containing LB liquid culture medium that has a volume of about 3 ml, afterwards, the test tube is immobilized on a shaker for shaking culture at 37° C. for 10 hours or overnight. The plasmids, which are extracted from the bacterial solution using an alkaline extraction method, are identified by a PCR method. The plasmids identified as positive undergo sequencing, and compared using DNAstar sequence analysis software. It turns out that the recombinant plasmids PBD-ZJCIVHA and PBD-ZJCIVNA are constructed successfully, the sequence of the HA gene is shown in SEQ ID NO.3 and the sequence of the NA gene is shown in SEQ ID NO.4.

### EXAMPLE 2

### Rescue of the Recombinant PR8 Virus

1. Preparation for Plasmid Transfection

Plasmids are extracted using an ultra-pure plasmid extraction kit (OMEGA), and the operation steps are as follows: 1) dip preserved glycerin bacteria (containing plasmids PBD-ZJCIVHA, PBD-ZJCIVNA, PBD-PR8M, PBD-PR8PB1, PBD-PR8PB2, PBD-PR8PA, PBD-PR8NS and PBD-PR8NP, and the last six plasmids are preserved in the lab) using an inoculating loop, draw lines on the surface of an Amp-containing LB flat plate, and stand overnight at 37° C.; 2) pick out and inoculate a single colony to a 5 ml Amp LB culture medium, carry out shaking culture at 37° C. until the OD600 value is 1.0-1.5; 3) collect 3 ml of overnight culture and centrifuge the culture to remove the culture medium thoroughly; 4) bacteria suspension: carry out bacteria suspension using 0.25 ml of RNase A-containing SolutionI until a homogenate state is reached; 5) cell lysis: add 0.25 ml of SolutionII, reverse 5 times to achieve gentle and uniform mixing; 6) neutralization: add 0.125 ml of Buffer N3, reverse 5 times immediately to achieve gentle and uniform mixing until white flocculent precipitates are formed, and centrifuge for 10 minutes at a rate of 12000×g under room temperature; 7) carefully pour the supernatant into a clean 1.5 ml centrifuge tube, add to the supernatant an ETR Solution (blue) the volume of which is one-tenth of that of the centrifuge tube, reverse 7-10 times, and put in an ice bath for 10 minutes; 8) add a water bath at 42° C. for 5 minutes, then mix to reach a turbid state, and centrifuge for 3 minutes at a rate of 12000×g under room temperature, so as to form a blue layer in the ETR solution at the bottom of the centrifuge tube; 9) transfer the supernatant into a new 1.5 ml centrifuge tube, add absolute ethyl alcohol the volume of which is a half of that of the centrifuge tube, reverse 6-7 times, and stand for 1-2 minutes under room temperature; 10) pour the aforementioned mixed solution into a HiBand DNAMini column that is properly balanced by 2ml of E4 in advance, put the column on a 2 ml collection tube, and centrifuge for 1 minute at a rate of 10000×g under room temperature so that lysis solution passes by the column; 11) discard the liquid in the collection tube, add the remaining mixed solution into the column, and centrifuge for 1 minute at a rate of 10000×g under room temperature so that lysis solution totally passes through the column; add 500 ul of Buffer HB to the column, centrifuge for 1 minute at a rate of 10000×g under room temperature, and wash the column to ensure removal of the residual proteins, in order to obtain high-quality DNA; 12) discard the liquid, wash the column with 700 ul of DNA Wash buffer, centrifuge for 1 minute at a rate of 10000×g under room temperature, and discard the liquid; 13) repeat the operation and then add DNA Wash buffer; 14) discard the liquid, carry out idling, centrifuge for 2 minutes at a rate of 12000×g under room temperature, and discard the liquid; 15) put the column in a clean 1.5 ml centrifuge tube, add 30-50 ul of endotoxin-free eluant onto the column, stand for 2 minutes under room temperature, and centrifuge for 1 minute at a rate of 12000×g under room temperature to elute DNA, wherein this elution can be carried out twice; and 16) carry out electrophoresis detection, and measure OD₂₆₀ and OD₂₈₀ using an Nanodrop 2000c ultraviolet-visible spectrophotometer to estimate DNA content and purity. Results: a sufficient amount of plasmids required by transfection are acquired.

2. Transfection of the 293T Cell

The aforementioned plasmids extracted at a ultra-purity, including PBD-ZJCIVHA, PBD-ZJCIVNA, PBD-PR8M, PBD-PR8PB1, PBD-PR8PB2, PBD-PR8PA, PBD-PR8NS and PBD-PR8NP, are co-transfected via a proper amount of liposome 2000 to a 293T cell having a diameter of 3.5 cm. 6 hours after transfection, the cell supernatant is discarded, 2 ml of OPTI-MEM (Invitrogen) culture solution is added, and the cell is put in a CO₂ incubator at 37° C. for culture for 72 hours.

3. Rescue of the Recombined PR8 Virus

48 hours after transfection, the cell supernatant is inoculated to a 9-day to 11-day SPF chicken embryo (BEIJING MERIAL VITAL LABORATORY ANIMAL TECHNOLOGY CO., LTD.), the chicken embryo is sealed by paraffin and then put in an incubator at 37° C. for culture. 48 to 72 hours later, the chicken embryo undergoes a temperature of 4° C. overnight, and then taken out to obtain chicken embryo allantoic fluid. The presence of hemagglutinin activity in the allantoic fluid is determined by a hemagglutination test. Results: hemagglutination occurs in the allantoic fluid, indicating that a PR8 recombinant virus containing the HA and NA genes of the ZJCIV has been rescued successfully.

4. Identification of the Recombinant Virus

The total RNA of the allantoic fluid of the recombinant virus is extracted using Trizol, and then subjected to reverse transcription by a 12-bp primer to obtain first-strand cDNA. The HA and NA fragments of the ZJCIV are PCR-amplified by taking the first-strand cDNA as the template and taking sapI-HA-up, sapI-HA-down and sapI-NA-up, sapI-NA-down as upstream and downstream primers, and then identified by 1% agarose electrophoresis. In addition, the ZJCIV HA and ZJCIV NA PCR products undergo sequencing in the company. Results: on agarose electrophoresis gel, there are two strips having a size of about 1700 bp and a size of about 1400 bp respectively, which are completely consistent with the target sizes. And it is further proved by sequencing results that these PCR products are indeed the HA and NA fragments of the ZJCIV.

### EXAMPLE 3

### Identification of the Growth Characteristics of the Rescued Recombinant Virus

1. Determination of EID₅₀ of the Rescued Recombinant Virus and the ZJCIV

The virus-containing chicken embryo allantoic fluid undergoes 10-fold dilutions, and the chicken embryo allantoic fluids that are diluted based on the dilutabilities from 10⁻⁶ to 10⁻¹⁰ are respectively inoculated to five 9-day to 11-day SPF chicken embryos for continuous incubation for 48 hours at 37° C. Whether the chicken embryo allantoic fluids are infected is judged by determining the hemagglutinin activities of the infected embryo allantoic fluids, and EID₅₀ (chicken embryos' median infective doses) is calculated using a Reed-Muench method. Results: EID₅₀ of the rescued recombinant virus and the ZJCIV are 10^{7.5}/100 ul and 10^{6.5}/100 ul, respectively.

2. Determination of TCID₅₀ of the Rescued Recombinant Virus and the ZJCIV

10-fold dilutions start from 1: 10⁻³, the recombinant virus and ZJCIV having different dilutabilities are inoculated to a 48-well plate on which monolayer MDCK cells grow, and the inoculation procedure is as follows: at first, clean the MDCK cells twice with PBS, then add 100 ul of virus to each well, repeat this operation 3 times for every dilutability, put the 48-well plate in a CO₂ incubator at 37° C. for the purpose that viruses are adsorbed on the cells, shake the cells left and right once at an interval of 20 minutes, discard the viruses in the cell culture plate 1.5-2.5 hours later, wash the cells twice with PBS, and then add 300 ul of serum-free medium. Continuously culture the virus-adsorbed cells in the CO₂ incubator for 72 hours, then determine the hemagglutinin activity of every well, and calculate TCID₅₀ (tissue cells' median infective doses) using a Reed-Muench method. Results: TCID₅₀ of the rescued recombinant virus and the ZJCIV are 10^{6.5}/100 ul and 10^{5.5}/100 ul, respectively.

3. Growth Characteristic Comparison of the Rescued Recombinant Virus and the ZJCIV on the Chicken Embryos

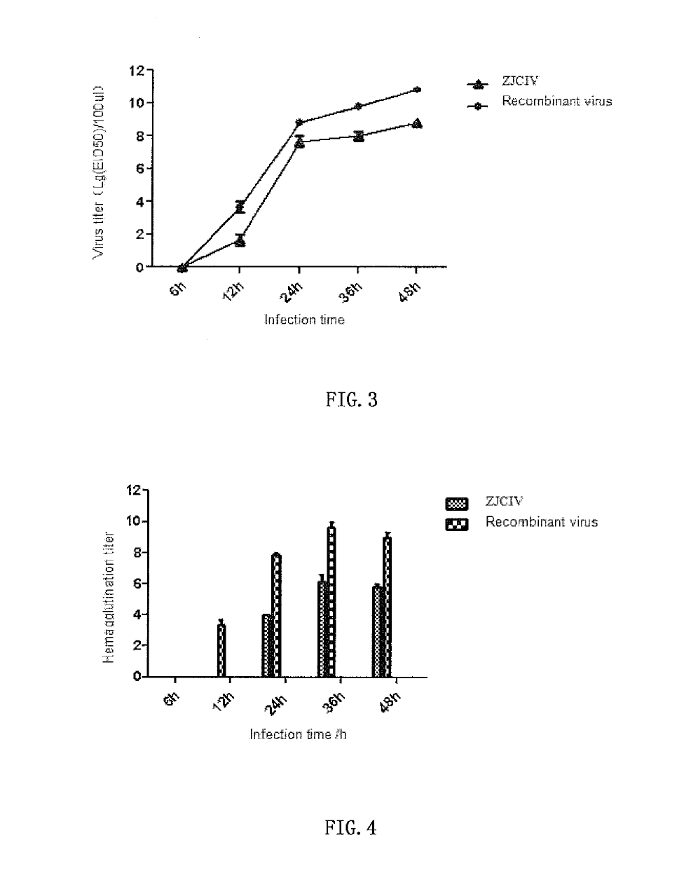

The rescued recombinant virus and the ZJCIV are diluted as 100EID₅₀, and are inoculated, based on this dilutability, to 18 9-day to 11-day SPF chicken embryos, respectively. 6 hours, 12 hours, 24 hours, 36 hours, 48 hours and 72 hours after inoculation, 3 inoculated SPF chicken embryos are taken out respectively, their allantoic fluids are collected and the hemagglutinin activities thereof are determined (FIG. 2). The allantoic fluids collected at different time undergo 10-fold dilutions, the virus solution of each dilutability is inoculated to 3 9-day to 11-day SPF chicken embryos, and the inoculum dose is 100 ul per chicken embryo. 48 hours after inoculation, the hemagglutinin activities of the chicken embryo allantoic fluids are determined, the virus contents of the allantoic fluids collected at different time are calculated, a growth curve of the virus is drawn up (FIG. 3), and the growth conditions of the rescued recombinant virus and the ZJCIV on the chicken embryos are compared. The results are shown in FIG. 2: the allantoic fluids of both the ZJCIV and the recombinant virus have no hemagglutination within 12 hours after inoculation. 48 hours after inoculation, the hemagglutination titer of the recombinant virus reaches 2¹¹, which is significantly higher than that of the ZJCIV. The results are shown in FIG. 3: no virus is detected 6 hours after inoculation of the ZJCIV and the rescued recombinant virus. 12 hours after inoculation, the virus titer of the rescued recombinant virus is significantly higher than that of the ZJCIV, and reaches the peak 48 hours after inoculation.

4. Growth Characteristic Comparison of the Rescued Recombinant Virus and the ZJCIV on the MDCK cells

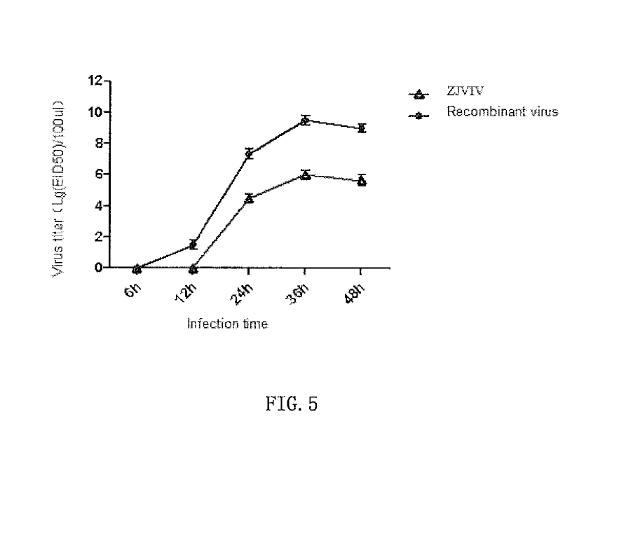

The rescued recombinant virus and the ZJCIV are diluted as 100TCID₅₀, and are each inoculated, based on this dilutability, to 3 T25 cell bottles in which 80% of the MDCK cells grow. 6 hours, 12 hours, 24 hours, 36 hours, 48 hours and 72 hours after inoculation, their cell supernatants are collected respectively and the hemagglutination titers thereof are determined (FIG. 4). The virus contents in the cell supernatants collected at different time are titrated, specifically as follows: the collected cell supernatants undergo 10-fold dilutions, the virus solution of each dilutability is inoculated to the MDCK cells with 80% growing in 3 wells of a 24-well cell plate, the hemagglutinin activities are determined 48 hours after inoculation, the virus contents (TCID₅₀) in the cell supernatants collected at different time are calculated, a growth curve of the virus is drawn up according to this TCID₅₀ (FIG. 5), and the growth conditions of the rescued recombinant virus and the ZJCIV on the MDCK cells are compared. The hemagglutination titer results are shown in FIG. 4: within 12 hours after inoculation, the cell supernatant of the ZJCIV has no hemagglutinin activity, while the cell supernatant of the recombinant virus has hemagglutinin activity, which reaches the peak 36 hours after inoculation and subsequently tends to a relatively stable state. In the entire infection process, the hemagglutinin activity of the recombinant virus is significantly higher than that of the ZJCIV. The virus titer results are shown in FIG. 5: no virus is detected 6 hours after inoculation of the ZJCIV and the rescued recombinant virus, and 12 hours after inoculation, the virus of the rescued recombinant virus can be detected, whereas the virus of the ZJCIV cannot be detected until 24 hours after inoculation. 36 hours after inoculation, the virus titers of the recombinant virus and the ZJCIV reach the peak and then begin descending. In the entire cell infection process, the virus titer of the recombinant virus is significantly higher than that of the ZJCIV.

The embodiments described above are for illustrating the ways of practicing the present invention only, and shall not be understood as limiting the patent scope of the present invention even if their descriptions are specific and detailed. It shall be noted that, many modifications and improvements could also be made by those ordinary skilled in this art without departing from the concept of the present invention, and these modifications and improvements shall fall within the scope of the present invention. Accordingly, the patent scope of the present invention shall be subject to the claims appended.

1. A canine influenza recombinant virus comprising HA and NA genes of ZJCIV canine influenza virus as well as six internal genes PA, PB1, PB2, M, NP and NS of a PR8 virus,
wherein the nucleotide sequence of the HA gene of the ZJCIV canine influenza virus is selected from the group consisting of:
(1) a nucleotide sequence encoding the amino acid sequence of SEQ ID NO.1;
(2) a nucleotide sequence encoding an amino acid sequence which has at least 98% sequence identity to the amino acid sequence of SEQ ID NO.1;
wherein the nucleotide sequence of the NA gene of the ZJCIV canine influenza virus is selected from the group consisting of:
(1) a nucleotide sequence encoding the amino acid sequence of SEQ ID NO.2;
(2) a nucleotide sequence encoding an amino acid sequence which has at least 98% sequence identity to the amino acid sequence of SEQ ID NO.2.

2. The canine influenza recombinant virus according to claim 1, wherein the HA gene of the ZJCIV canine influenza virus has the nucleotide sequence of SEQ ID NO.3, or the HA gene of the ZJCIV canine influenza virus has a sequence having at least 98% sequence identity to the nucleotide sequence of SEQ ID NO.3.

3. The canine influenza recombinant virus according to claim 1, wherein the NA gene of the ZJCIV canine influenza virus has the nucleotide sequence of SEQ ID NO.4, or the NA gene of the canine influenza virus has a sequence having at least 98% sequence identity to the nucleotide sequence of SEQ ID NO.4.

4. A method for preparing a canine influenza recombinant virus, the method comprising:
constructing recombinant plasmids comprising the HA and NA genes of the ZJCIV canine influenza virus, respectively;
transfecting the recombinant plasmids of the HA and NA genes, and six plasmids comprising the internal genes PA, PB1, PB2, M, NP and NS of the PR8 virus respectively, to a 293T cell, and culturing the transfected cell;
inoculating a chicken embryo with a supernatant of the cultured cell, culturing the chicken embryo in an incubator for a selected duration to obtain a chicken embryo allantoic fluid, detecting a hemagglutination condition of the allantoic fluid, and at the presence of hemagglutinin activity, determining the absence of unexpected variations by sequencing to obtain the canine influenza recombinant virus.

5. The method according to claim 4, wherein the recombinant plasmids contain PBD vectors used as empty vectors.

6. The method according to claim 4, wherein the supernatant of the cultured cell is inoculated to a 9-day to 11-day chicken embryo, and the chicken embryo is cultured in a 37° C. incubator for 48-72 hours to obtain the chicken embryo allantoic fluid.

7. A method for preventing or treating canine influenza, comprising administering to a canine in need thereof the canine influenza recombinant virus according to claim 1.

8. An influenza vaccine comprising the canine influenza recombinant virus as claimed in claim 1.

9. The influenza vaccine according to claim 8, wherein the influenza vaccine further comprises adjuvant.

10. A method of preventing or treating canine influenza, the method comprising administering the influenza vaccine as claimed in claim 8 to a subject.


SEQUENCE LISTING (9 sequences)

SEQ ID NO: 1 - protein, 566 aa, canine influenza virus
MKTVIALSYIFCLAFGQNLPGNENNAATLCLGHHAVPNGTIVKTITDDQIEVTNATELVQNSPTGKICNNPHKILDGRDCTLIDALLGDPHCDVFQNETWDLFVERSNAFSNCYPYDVPDYASLRSIVASSGTLEFITEGFTWAGVTQNGGSGACKRGPANGFFSRLNWLTKSGNTYPVLNVTMPNNNNFDKLYIWGVHHPSTNQEQTSLYIQASGRVTVSTRRSQQTIIPNIGSRPLVRGQSGRISVYWTIVKPGDVLVINSNGNLIAPRGYFKMHIGKSSIMRSDAPIDTCISECITPNGSIPNEKPFQNVNKITYGACPKYVKQNTLKLATGMRNVPERQTRGLFGAIAGFIENGWEGMVDGWYGFRHQNSEGTGQAADLKSTQAAIDQINGKLNRMIEKTNEKFHQIEKEFSEVEGRIQDLERYVEDTKVDLWSYNAELLVALENQNTIDLTDSEMNKLFEKTRRQLRENAEDMGNGCFKIYHKCDNACIESIRNGTYDHNIYRDEAVNNRFQIKGVELKSGYKDWILWISFAISCFLLCVVLLGFIMWACQRGNIRCNICI

SEQ ID NO: 2 - protein, 471 aa, Canine influenza virus
MNPNQKIIAIGSVSLTIATVCFLLQIAILATTVTLYFKQNECNIPSNSQVVPCKPIIIERNITEVVYLNNTTIEKEKEICSVVLEYRNWSKPQCQITGFAPFSKDNSIRLSAGGDIWVTREPYVSCDPSKCYQFALGQGTTLNNKHSNGTIHDRISHRTLLMNELGVPFHLGTKQVCIAWSSSSCHDGKAWLHVCVTGDDRNATASFVYNGMLVDSIGSWSQNILRTQESECVCINGTCTVVMTDGSASGRADTRILLIREGKIVHISPLSGSAQHIEECSCYPRYPNVRCVCRDNWKGSNRPVIDINMADYSIDSSYVCSGLVGDTPRNDDSSSSSNCRDPNNERGNPGVKGWAFDNENDVWMGRTISRNLRSGYETFKVIGGWTTANSKSQVNRQVIVDNNNWSGYSGIFSVEGKSCVNRCFYVELIRGGPQETRVWWTSNSIVVFCGTSGTYGTGSWPDGANINFMPI

SEQ ID NO: 3 - DNA, 1701 bp, canine influenza virus
atgaaaactgttattgctttaagctacattttctgcctggcttttggtcagaatcttccaggaaatgaaaataatgctgcaacactatgcctgggacatcatgcagtgccgaacgggacaatagtgaaaactatcacagacgatcaaattgaggtgaccaacgccaccgagctagtccaaaactccccaacagggaaaatatgcaacaatccccacaagattcttgatgggagggactgcacactaatagatgccctactaggggacccgcactgtgacgtcttccaaaatgagacatgggacctttttgtggaacgaagcaatgcttttagcaattgttacccttatgatgtaccagactatgcatcccttcgatccatagttgcatcatcaggcacattggagttcatcactgaaggtttcacttgggcaggagtaactcaaaatggaggaagcggtgcttgtaaaaggggacctgctaatggtttcttcagtagattgaattggttaactaagtcaggaaatacatatccagtgttgaatgtgactatgccaaacaataacaatttcgacaaattatacatttggggagttcatcacccaagcactaatcaagaacaaaccagcctgtatattcaggcctcaggaagagtcacagtctctaccaggagaagccaacagaccataatcccaaacattggatctagacccttggtaaggggccaatctggcagaataagcgtatattggacaatagtcaaacctggagacgtactggtaataaacagtaatggaaacctaatcgctcctcgaggctacttcaaaatgcacattgggaaaagctcaataatgagatcagatgcgcctattgacacctgcatttccgaatgtatcactccgaacgggagcatccccaatgaaaagcccttccaaaatgtgaacaagatcacatacggagcatgtcccaaatatgttaagcaaaacaccttgaaactggcaacaggaatgcggaatgtccctgagaggcaaaccagaggcctgttcggcgcaatagcaggcttcatagaaaatggatgggaagggatggtagacggttggtatggcttcaggcaccaaaattccgaaggtacaggacaagcagcagaccttaaaagcactcaggcagccattgaccagattaatgggaaattgaacagaatgattgaaaaaacgaatgagaagttccatcaaatcgaaaaggagttttccgaagtagaagggaggattcaagaccttgagagatacgttgaagacacaaaagtagatctttggtcttacaatgccgagcttcttgttgctttagaaaaccagaacacaattgatttaactgattcagaaatgaacaaattgtttgaaaagactaggaggcaattgagggaaaatgctgaagacatgggcaatggctgcttcaagatataccacaagtgtgacaatgcttgcatagaatcgattagaaacggaacttatgaccataacatatatagagatgaggcagtgaacaatcggttccagatcaaaggtgttgagctaaagtctggatacaaagactggatcttgtggatttcctttgccatatcatgctttttgctttgtgttgtcttgctgggtttcattatgtgggcctgccagagaggcaacattaggtgcaacatttgcatttga

SEQ ID NO: 4 - DNA, 1416 bp, canine influenza virus
atgaacccaaatcaaaagataatagcaataggttctgtctctctaaccattgcaacagtatgtttcctcttgcagattgccatcctagcaacaactgtgacactgtacttcaagcaaaatgaatgcaacatcccctcgaacagtcaagtagtgccatgtaaaccaatcataatagaaaggaacataacagaggtagtatatttgaataatactaccatagaaaaagaaaaagaaatttgttccgtagtgctagaatacaggaactggtcgaaaccgcagtgtcaaattacaggatttgctcctttctccaaggacaactcaatccgactctccgctggtggggacatttgggtaacaagggaaccttatgtgtcatgcgaccccagcaaatgttatcagtttgcacttgggcaggggaccacgctgaacaataaacactcaaacggcacaatacatgataggatctctcatcgaactcttttaatgaatgagttgggtgttccgtttcatttgggaaccaaacaagtgtgcatagcatggtccagttcaagttgtcacgatgggaaagcatggttacatgtttgtgtcactggggatgatagaaatgcgactgctagtttcgtttataatggaatgcttgttgacagtattggttcatggtctcaaaatatcctcagaactcaagagtcagaatgcgtttgcatcaatggaacttgtacagtagtaatgactgatggaagtgcatcaggaagggctgatactagaatactactcatcagagaggggaaaattgtccatattagcccattgtcagggagtgctcaacatatagaggaatgttcctgttatcctcgatatccaaatgttagatgtgtttgcagagacaattggaagggctctaataggcccgttatagatataaatatggcagattatagcatcgattccagttatgtgtgttcaggacttgttggcgatacaccaaggaatgatgatagctctagcagcagtaactgcagggatcctaataatgagagagggaatccaggagtgaaagggtgggcctttgataatgagaatgacgtttggatggggaggacaatcagcagaaatttgcgctcaggttatgagactttcaaggtcattggtggctggaccactgctaattccaagtcacaggtcaatagacaagtcatagttgacaataataactggtctggttattctggtattttctccgttgaaggcaaaagctgtgttaataggtgtttttatgtagagttgataagaggagggccacaagagactagagtatggtggacttcaaatagcattgtcgtattttgtggtacttctggtacctatggaacaggctcatggcctgatggggcgaatatcaacttcatgcctatataa

SEQ ID NO: 5 - DNA, 31 bp, Artificial Sequence
  Synthetic
cacacagctcttctattagcaaaagcagggg

SEQ ID NO: 6 - DNA, 37 bp, Artificial Sequence
  Synthetic
cacacagctcttcggccagtagaaacaagggtgtttt

SEQ ID NO: 7 - DNA, 32 bp, Artificial Sequence
  Synthetic
cacacagctcttctattagcaaaagcaggagt

SEQ ID NO: 8 - DNA, 38 bp, Artificial Sequence
  Synthetic
cacacagctcttcggccagtagaaacaaggagtttttt

SEQ ID NO: 9 - DNA, 12 bp, Artificial Sequence
  Synthetic
agcaaaagcagg